(19) United States
(12) Patent Application Publication
(10) Pub. No.: US 2026/0248929 A1
(43) Pub. Date: Aug. 27, 2026

(54) MINOXIDIL-CROSSLINKED INJECTABLE HYDROGELS FOR LOCAL AND SUSTAINED INTRADERMAL DELIVER Y

(71) Applicant: THE TRUSTEES OF THE UNIVERSITY OF PENNSYLVANIA, Philadelphia, PA (US)

(72) Inventors: Leo WANG, Philadelphia, PA (US); George COTSARELIS, Berwyn, PA (US)

(73) Assignee: THE TRUSTEES OF THE UNIVERSITY OF PENNSYLVANIA, Philadelphia, PA (US)

(21) Appl. No.: 19/163,146

(22) PCT Filed: Mar. 7, 2024
(86) PCT No.: PCT/US24/18977

§ 371 Date: Sep. 8, 2025

Related U.S. Application Data
(60) Provisional application No. 63/450,958, filed on Mar. 8, 2023.

(51) Int. Cl. A61K 47/61; A61K 9/06; A61P 17/14

(52) U.S. Cl. CPC A61K 47/61; A61K 9/06; A61P 17/14

(57) ABSTRACT

This invention provides a sustained delivery hydrogel comprising a crosslinked minoxidil-aldehyde-modified polymer for treatment of hair loss. Also provided herein is a sustained delivery hydrogel comprising a crosslinked minoxidil-aldehyde-modified hyaluronic acid derivative for treatment of hair loss. The invention also relates to methods for inducing anagen phase of hair growth, prolonging the anagen phase, increasing hair follicle size, reversing, preventing or inhibiting miniaturization of the hair follicle, and combinations thereof in a subject having hair loss, comprising administering the sustained delivery hydrogel comprising the crosslinked minoxidil-aldehyde-modified hyaluronic acid derivative to an area of hair loss of the subject, and to methods for inducing anagen phase of hair growth, prolonging the anagen phase, increasing hair follicle size, reversing, preventing or inhibiting miniaturization of the hair follicle, and combinations thereof in a subject having hair loss, comprising administering the sustained delivery hydrogel comprising the crosslinked minoxidil-aldehyde-modified polymer to an area of hair loss of the subject.

### FIELD OF THE INVENTION

The invention relates to sustained delivery hydrogels comprising a crosslinked minoxidil-aldehyde-modified polymer for treatment of hair loss and methods of producing and using the same for treatment of hair loss. The invention also relates to sustained delivery hydrogels comprising a crosslinked minoxidil-aldehyde-modified hyaluronic acid derivative for treatment of hair loss and methods of producing and using the same for treatment of hair loss. The invention also relates to methods for prolonging anagen in a subject having hair loss, comprising administering a sustained delivery hydrogel comprising a crosslinked minoxidil-aldchyde-modified hyaluronic acid derivative to an area of hair loss of the subject. The invention further relates to methods for prolonging anagen in a subject having hair loss, comprising administering a sustained delivery hydrogel comprising a crosslinked minoxidil-aldehyde-modified polymer to an area of hair loss of the subject. The invention further relates to methods for reversing, preventing or inhibiting hair follicle miniaturization in a subject having hair loss, comprising administering a sustained delivery hydrogel comprising a crosslinked minoxidil-aldehyde-modified polymer to an area of hair loss of the subject.

### BACKGROUND OF THE INVENTION

Androgenetic alopecia (AGA) is thought to affect at least half of men and a third of women by the age of 50. Currently, the mainstay of treatment for androgenetic alopecia is minoxidil (ROGAINE®), which is a small molecule that is delivered topically to the skin for androgenetic alopecia and other forms of hair loss. Minoxidil is a repurposed antihypertensive that is thought to promote vasodilation, induce hair follicles to enter anagen, the growth phase of the hair cycle, and reverse miniaturization of hair follicles. To be effective, minoxidil must be applied once to twice daily for several months. Adherence to this regimen is a major barrier to treatment. Moreover, topical penetrance of minoxidil is limited and only 1% of minoxidil is absorbed from a topical application.

Minoxidil can be irritating to the skin, cause reddened skin, burning of the scalp, swelling of the face, folliculitis and dermatitis at the site of application, give existing hair a greasy look, and/or increase facial hair growth. Moreover, the effect of minoxidil ceases when administration is discontinued.

Oral administration of minoxidil produces adverse effects, such as systemic toxicities, including hypotension, edema, pericardial effusions, and hyponatremia. Other reported harmful side effects are weight gain, fast heartbeat and chest pain. Another undesirable side effect of oral minoxidil is excessive hair growth in undesired areas, which has been a major limitation for the use of oral minoxidil by hair loss patients, especially women.

Accordingly, there remains a need for developing improved compositions and methods for treatment of hair loss by inducing and prolonging anagen, and reversing and preventing miniaturization.

### SUMMARY OF THE INVENTION

In one aspect, provided herein is a sustained delivery hydrogel comprising a crosslinked minoxidil-aldehyde-modified polymer for treatment of hair loss.

In another aspect, provided herein is a sustained delivery hydrogel comprising a crosslinked minoxidil-aldehyde-modified hyaluronic acid derivative for treatment of hair loss.

In another aspect, provided herein is a sustained delivery hydrogel comprising a crosslinked minoxidil-aldehyde-modified hyaluronic acid derivative for treatment of hair loss having chemical structure I:

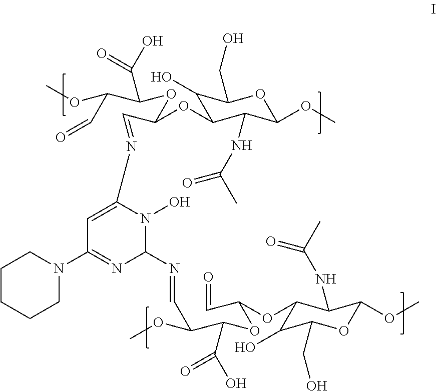

In another aspect, provided herein is a method for producing a sustained delivery hydrogel comprising a crosslinked minoxidil-aldehyde-modified polymer, the method comprising: preparing an aldehyde-modified polymer; dissolving the aldehyde-modified polymer in a minoxidil solution comprising phosphate buffered saline; and mixing the minoxidil solution comprising the dissolved aldehyde-modified polymer to react aldehydes of the aldehyde-modified polymer and primary amines of the minoxidil, thereby forming the hydrogel.

In still another aspect, provided herein is a method for producing a sustained delivery hydrogel comprising a crosslinked minoxidil-aldehyde-modified hyaluronic acid derivative, the method comprising: preparing an aldehyde-modified hyaluronic acid derivative; dissolving the aldehyde-modified hyaluronic acid derivative in a minoxidil solution comprising phosphate buffered saline; and mixing the minoxidil solution comprising the dissolved aldehyde-modified polymer to react aldehydes of the aldehyde-modified polymer and primary amines of the minoxidil, thereby forming the hydrogel.

In still another aspect, provided herein is a method for producing a sustained delivery hydrogel comprising a crosslinked minoxidil-aldehyde-modified hyaluronic acid derivative, the method comprising: preparing an aldehyde-modified hyaluronic acid derivative; dissolving the aldehyde-modified hyaluronic acid derivative in a minoxidil solution comprising phosphate buffered saline; and mixing the minoxidil solution comprising the dissolved aldchyde-modified polymer to react aldehydes of the aldehyde-modified polymer and primary amines of the minoxidil, thereby forming the hydrogel.

In another aspect, provided herein is a method for treating hair loss in a subject in need thereof, the method comprising administering a sustained delivery hydrogel comprising a crosslinked minoxidil-aldehyde-modified hyaluronic acid derivative to an area of hair loss of the subject, wherein administration comprises: (a) transdermal administration comprising subcutaneous injection, microneedle array injection or application of a transdermal patch comprising the hydrogel; (b) local administration comprising topical application of an ointment, gel, nanoparticles or liposomes comprising the hydrogel; or (c) a combination of (i) skin needling, and (ii) the local administration of (b).

In another aspect, provided herein is a method for inducing anagen in a subject having hair loss, the method comprising administering a sustained delivery hydrogel comprising a crosslinked minoxidil-aldehyde-modified polymer to an area of hair loss of the subject, wherein administration comprises: (a) transdermal administration comprising subcutaneous injection, microneedle array injection or application of a transdermal patch comprising the hydrogel; (b) local administration comprising topical application of an ointment, gel, nanoparticles or liposomes comprising the hydrogel; or (c) a combination of (i) skin needling, and (ii) the local administration of (b).

In one aspect, provided herein is a method for inducing anagen in a subject having hair loss, the method comprising administering a sustained delivery hydrogel comprising a crosslinked minoxidil-aldehyde-modified hyaluronic acid derivative to an area of hair loss of the subject, wherein administration comprises: (a) transdermal administration comprising subcutaneous injection, microneedle array injection or application of a transdermal patch comprising the hydrogel; (b) local administration comprising topical application of an ointment, gel, nanoparticles or liposomes comprising the hydrogel; or (c) a combination of (i) skin needling, and (ii) the local administration of (b).

In one aspect, provided herein is a method for reversing, preventing or inhibiting miniaturization of the hair follicle in a subject having hair loss, the method comprising administering a sustained delivery hydrogel comprising a crosslinked minoxidil-aldehyde-modified hyaluronic acid derivative to an area of hair loss of the subject, wherein administration comprises: (a) transdermal administration comprising subcutaneous injection, microneedle array injection or application of a transdermal patch comprising the hydrogel; (b) local administration comprising topical application of an ointment, gel, nanoparticles or liposomes comprising the hydrogel; or (c) a combination of (i) skin needling, and (ii) the local administration of (b).

Other features and advantages of the present invention will become apparent from the following detailed description examples and figures. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present disclosure, the inventions of which can be better understood by reference to one or more of these drawings in combination with the detailed description of specific embodiments presented herein. The patent or application file contains at least one drawing executed in color. Copies of this patent or patent application publication with color drawing(s) will be provided by the Office upon request and payment of the necessary fee.

FIGS. 1A-1B shows hydrogel chemistry and delivery. Aldehyde groups are formed from the cleavage of cis-glycols on hyaluronic acid by sodium periodate (FIG. 1A). When aldehyde-modified hyaluronic acid is mixed with minoxidil, a reaction between aldehydes and the primary amines of minoxidil forms a Schiff-base, which is a dynamic covalent bond (FIG. 1B), forming a hydrogel that was shown to be capable of injection with excellent retention in vivo. Upon injection into the scalp, Schiff bases react with water and reverse the reaction to release minoxidil from the hydrogel where it can act to induce anagen and/or prolong anagen and/or reverse, prevent or inhibit miniaturization of the hair follicle.

FIG. 2 shows a sustained delivery hydrogel comprising a crosslinked minoxidil-aldehyde-modified hyaluronic acid derivative (“minoxidil hydrogel”) according to embodiments of the present invention. Minoxidil hydrogels are rapidly assembled from mixing of minoxidil with aldchyde modified hyaluronic acid.

FIGS. 3A-3D show anagen induction by a minoxidil hydrogel in telogen mice at 12 days. Telogen mice at eight weeks had Nair™ applied to dorsal skin and were subsequently injected with either an aldehyde-modified hyaluronic acid alone (control) (FIG. 3A), or hydrogels crosslinked with minoxidil (FIG. 3B) or minoxidil sulfate at varying concentrations (FIGS. 3C-3D). Two five microliter injections of gel were injected superiorly (red boxes). Local anagen induction was observed in multiple mice after 12 days (red arrows), which was seen only in the treatment groups.

FIGS. 4A-4C show the determining of minoxidil release by UV spectroscopy. Minoxidil absorbance is shown in FIGS. 4A-4B; minoxidil release is shown in FIG. 4C.

FIGS. 5A-5F show the preparation and application of microneedles patches according to an embodiment of the present invention. FIG. 5A shows a customizable microneedle mold. (stainless steel, microscale 3D printing. FIG. 5B shows a silicone cured over mold. FIG. 5C shows a silicone microneedle template. FIG. 5D shows a polymer and drug cast into a template to form hydrogels. FIG. 5E shows centrifugation and dehydration of the hydrogel. FIG. 5F shows application of a microneedles patch to the skin.

FIGS. 6A-6B show microneedle patches (10×10, 1 cm2) according to an embodiment of the present invention with light microscopy.

FIGS. 7A-7D show microneedle patches (10×10, 1 cm2) according to an embodiment of the present invention with red dye for contrast.

FIGS. 8A-8C show minoxidil microneedle patches in combination applied to mice in telogen (~8 weeks old) after clipping hair and then occluded under a transparent medical dressing (Tegaderm™) (FIG. 8A) induce anagen after 10 days (FIGS. 8B-8C).

### DETAILED DESCRIPTION OF THE INVENTION

The present subject matter may be understood more readily by reference to the following detailed description which forms a part of this disclosure. It is to be understood that this invention is not limited to the specific products, methods, conditions or parameters described and/or shown herein, and that the terminology used herein is for the purpose of describing particular embodiments by way of example only and is not intended to be limiting of the claimed invention.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

As employed above and throughout the disclosure, the following terms and abbreviations, unless otherwise indicated, shall be understood to have the following meanings.

In the present disclosure the singular forms “a,” “an,” and “the” include the plural reference, and reference to a particular numerical value includes at least that particular value, unless the context clearly indicates otherwise. Thus, for example, a reference to “a compound” is a reference to one or more of such compounds and equivalents thereof known to those skilled in the art, and so forth. The term “plurality”, as used herein, means more than one. When a range of values is expressed, another embodiment incudes from the one particular and/or to the other particular value. Similarly, when values are expressed as approximations, by use of the antecedent “about,” it is understood that the particular value forms another embodiment. All ranges are inclusive and combinable.

As used herein, the terms “component,” “composition,” “composition of compounds,” “compound,” “drug,” “pharmacologically active agent,” “active agent,” “therapeutic,” “therapy,” “treatment,” or “medicament” are used interchangeably herein to refer to a compound or compounds or composition of matter which, when administered to a subject (human or animal) induces a desired pharmacological and/or physiologic effect by local and/or systemic action.

As used herein, the terms “treatment” or “therapy” (as well as different forms thereof) include preventative (e.g., prophylactic), curative or palliative treatment. As used herein, the term “treating” includes alleviating or reducing at least one adverse or negative effect or symptom of a condition, disease or disorder.

The terms “subject,” “individual,” and “patient” are used interchangeably herein, and refer to an animal, for example a human, to whom treatment, including prophylactic treatment, with the pharmaceutical composition according to the present invention, is provided. The term “subject” as used herein refers to human and non-human animals. The terms “non-human animals” and “non-human mammals” are used interchangeably herein and include all vertebrates, e.g., mammals, such as non-human primates, (particularly higher primates), sheep, dog, rodent, (e.g., mouse or rat), guinea pig, goat, pig, cat, rabbits, cows, horses and non-mammals such as reptiles, amphibians, chickens, and turkeys.

Injectable hydrogels are crosslinked polymer networks that can be used to encapsulate therapeutics for local and sustained delivery. Sustained intradermal delivery of minoxidil from a hydrogel can overcome many of the current limitations to treatment. However, small molecules, such as minoxidil, are traditionally challenging to deliver due to their small size, which diffuses freely out of hydrogels and into surrounding tissue. Few hydrogel formulations of minoxidil have been explored, none of which have had a measurable effect clinically.

As used herein, “crosslinking” means chemically joining two or more molecules by a covalent bond. A “crosslinking reagent” or a “crosslinker”, which are used interchangeably herein means a molecule that links an aldehyde-modified polymer, including but not limited to an aldehyde-modified hyaluronic acid, that forms a crosslink (i.e., a bond) between the aldehyde groups and the primary amines of minoxidil through a Schiff base reaction, i.e., a reaction between amine groups and aldehyde groups, resulting in imine bonds, i.e., double bonds between carbon and nitrogen (—C═N—). Aldehyde groups are formed by the cleavage of cis-glycols on hyaluronic acid by sodium periodate. In an exemplary embodiment, when aldehyde-modified hyaluronic acid is mixed with minoxidil, a reaction between the aldehydes of the aldehyde-modified hyaluronic acid and the primary amines of minoxidil forms a Schiff-base, which is a dynamic covalent bond, thereby forming a hydrogel.

As used herein, “sustained delivery” means a slow drug delivery that is maintained over an extended period of time. As used herein, a “sustained delivery hydrogel,” a “sustained delivery hydrogel comprising a crosslinked minoxidil-aldehyde-modified polymer” and a “sustained delivery hydrogel comprising a crosslinked minoxidil-aldehyde-modified hyaluronic acid derivative,” which are used interchangeably herein, mean a hydrogel comprising a crosslinked minoxidil-aldehyde-modified polymer that delivers the active pharmaceutical ingredient (API) minoxidil from the hydrogel means over 4 to 6 weeks after administration to the subject, wherein administration comprises transdermal administration or local administration, as described herein and as commonly understood by those of ordinary skill in the art. In an exemplary embodiment, the sustained delivery hydrogel comprises a minoxidil-aldehydic-modified hyaluronic acid derivative that delivers the API minoxidil from the hydrogel over 4 to 6 weeks after administration to the subject.

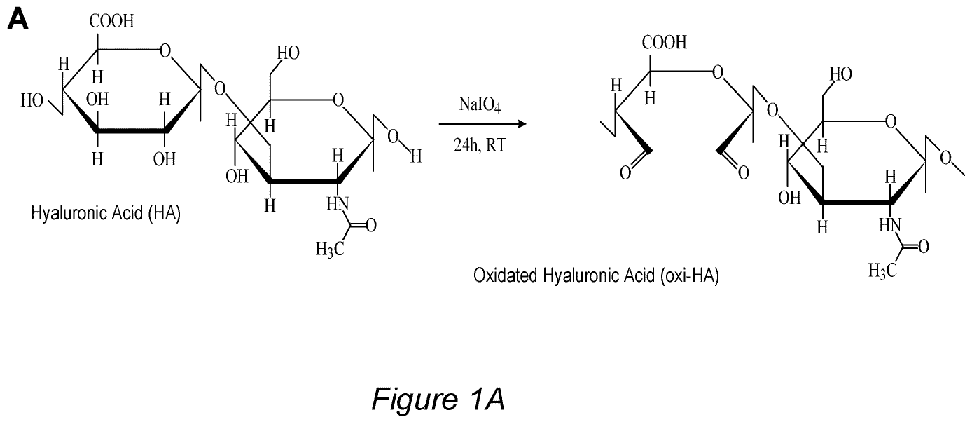

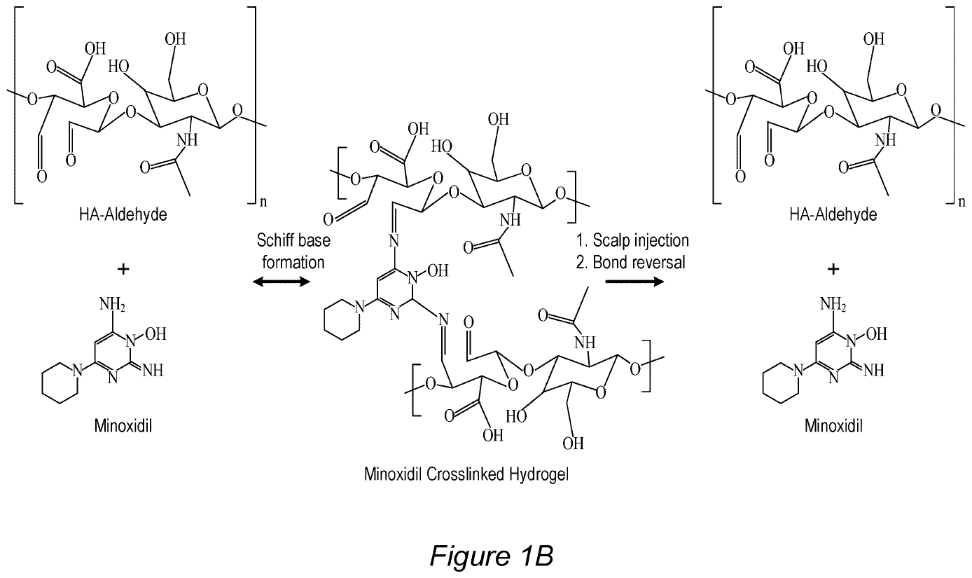

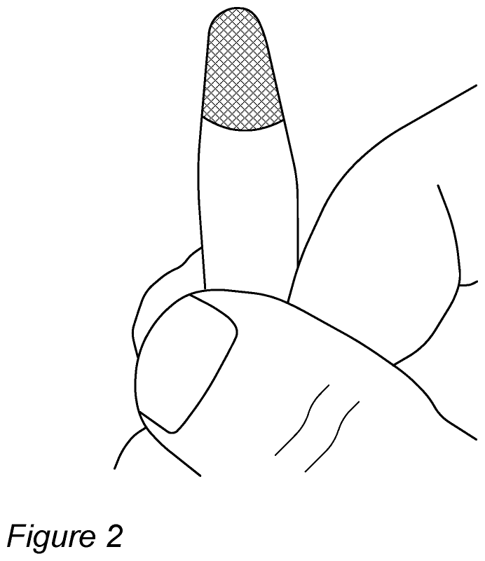

To overcome this challenge, it was observed that minoxidil has two primary amine groups that make it chemically reactive with aldehydes and it was hypothesized that aldehyde-modified hyaluronic acid polymers could be crosslinked with minoxidil into hydrogels. Because minoxidil has two primary amine groups, it can form a crosslink between aldehyde groups through a Schiff base reaction. This crosslinking forms a hydrogel with unique properties. The Schiff base is a dynamic covalent bond, which covalently conjugates minoxidil to the backbone, allowing release to be sustained based on the stability of the bond. Dynamic covalent bonds also form shear-thinning and self-healing materials. When exposed to shear forces, crosslinks break, allowing the hydrogels to pass through a syringe. Once through the syringe, the broken crosslinks may self-heal, reforming bonds to stabilize the gels. Upon delivery into tissue, minoxidil is released slowly to the hair follicle (FIGS. 1A-1B). The successfully synthesized this material and hydrogel using multiple minoxidil formulations that can be injected through a 30-gauge needle (FIG. 2).

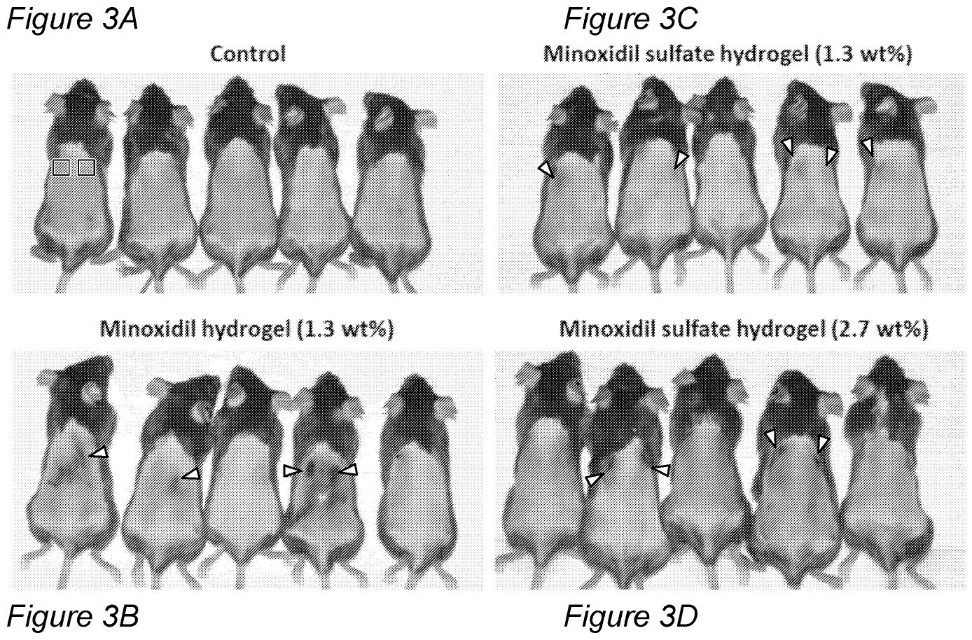

In the present case, hydrogel formulations of both minoxidil and minoxidil sulfate (active form in vivo) were tested in a mouse model of anagen induction. In this model, eight-week old mice in the telogen, resting phase, of the hair cycle received a chemical depilatory treatment and then had two hydrogel depots injected intradermally. Anagen induction was noted in multiple treatment groups, observed from the increased pigment in hair follicles surrounding injection sites twelve days post injection (FIGS. 3A-3D). There was no anagen induction found in control groups treated with aldehyde-modified hyaluronic acid without minoxidil. This therapeutic hydrogel represents a minimally invasive injectable option for the treatment of androgenetic alopecia, as well as other types of alopecia described herein, that overcomes many of the current compliance and absorption limitations that limit therapeutic efficacy. This therapeutic platform enables codelivery of other cofactors (i.e., finasteride, dutasteride); for example, coadministration of a separate composition comprising long-acting finasteride or dutasteride sequentially in any order or concurrently with the administration of the sustained delivery hydrogel comprising a crosslinked minoxidil-aldehyde-modified polymer according to embodiments of the invention, e.g., a sustained delivery hydrogel comprising a crosslinked minoxidil-aldehyde-modified hyaluronic acid derivative, may improve hair growth. Further, multiple carbohydrate-based polymers (e.g., cellulose, glycogen, amylose) can be modified with aldehydes and co-delivered with minoxidil, having two primary amine groups after crosslinking with an aldehyde-modified carbohydrate-based polymer, as an injectable hydrogel for local and sustained delivery. According to embodiments of the present invention, the minoxidil is the crosslinker. The properties conferred by the crosslinked minoxidil-aldehyde-modified polymer hydrogels, according to embodiments of the present invention include (i) shear-thinning that enables the hydrogels to pass through a needle and establishment of a depot in the dermis after injection, and (ii) sustained release of minoxidil.

Currently, minoxidil is applied once or twice daily to the scalp, which limits compliance to hair loss treatment. The sustained delivery hydrogels comprising a crosslinked minoxidil-aldehyde-modified polymer hydrogel, according to embodiments of the present invention, may be delivered as a one-time minoxidil hydrogel injection every two to three months and can sustain the intradermal release of the minoxidil molecule. In an embodiment, the crosslinked minoxidil-aldehyde-modified polymer hydrogel comprises a crosslinked minoxidil-aldehyde-modified hyaluronic acid derivative according to embodiments of the present invention. The injections are minimally painful and no anesthetics are necessary. The dermal injections may be administered at a clinic or at a doctor's office. Additionally, the sustained delivery hydrogels can be delivered through a multi-injector device to inject multiple small deposits of about 5-100 μL deposits of hydrogel across a large surface area of scalp comprising an area of 120 cm² of an average scalp at a time. In an embodiment, dermal injections of the provided crosslinked minoxidil-aldehyde-modified polymers may be administered to one or more bald spots, each having an area of about 3 centimeters (cm²). In an embodiment, an area of about 10 cm² is treated with a crosslinked minoxidil-aldehyde-modified polymer according to the present invention.

In one aspect, provided herein is a sustained delivery hydrogel comprising a crosslinked minoxidil-aldehyde-modified polymer for treatment of hair loss. In an embodiment, the sustained delivery hydrogel is formulated for transdermal administration or local administration, wherein the transdermal administration comprises subcutaneous injection, microneedle array injection or application of a transdermal patch comprising the hydrogel; wherein the local administration comprises topical application of an ointment, gel, nanoparticles or liposomes comprising the hydrogel. In another embodiment, the hair loss is androgenetic alopecia, alopecia areata, frontal fibrosing alopecia, diffuse alopecia, scarring alopecia or wherein the alopecia areata is universal alopecia (alopecia universalis). In certain embodiments, the microneedle array or the transdermal patch comprises a sustained delivery hydrogel comprising a crosslinked minoxidil-aldchyde-modified polymer and a polymer that produces a bulking effect and adds rigidity to the hydrogel. In an embodiment, the sustained delivery hydrogel comprises minoxidil, crosslinked HA-aldehyde and gelatin. In some embodiments, the hair loss is male or female pattern alopecia. In an embodiment of the sustained delivery hydrogel, the hydrogel comprises the aldehyde-modified polymer at a weight percent (wt. %) of from 1 wt. % to 10 wt. % and the minoxidil at wt. % of from 0.1 wt. % to 5 wt. %. In certain embodiments, the polymer is selected from the group consisting of collagen, elastin, thrombin, fibronectin, starches, poly(amino acid), poly(propylene fumarate), gelatin, alginate, pectin, fibrin, oxidized cellulose, chitin, chitosan, tropoelastin, hyaluronic acid, polyethylene, polyethylene terephthalate, poly(tetrafluoroethylene), polycarbonate, polypropylene and poly(vinyl alcohol), ribonucleic acids, deoxyribonucleic acids, polypeptides, proteins, polysaccharides, and polynucleotides. In an embodiment, the herein described sustained delivery hydrogel further comprises a second polymer that produces a bulking effect and adds rigidity to the hydrogel (“a bulking polymer”), wherein the bulking polymer is selected from the above-listed polymers. In certain embodiments, the bulking polymer comprises one or more of said bulking polymers selected from the above-listed polymers. In some embodiments, said bulking polymer produces a bulking effect and adds rigidity to a hydrogel in the microneedle array or in the transdermal patch. In an embodiment said bulking polymer is gelatin. In some embodiments, the hydrogel comprises from 0 wt. % to 50 wt. % of said bulking polymer. In certain embodiments, the hydrogel comprises from 1 wt. % to 50 wt. % of said bulking polymer. In some embodiments, the hydrogel comprises from 1 wt. % to 40 wt. % of said bulking polymer. In an embodiment, the hydrogel comprises from 1 wt. % to 30 wt. % of said bulking polymer. In various embodiments, the hydrogel comprises from 1 wt. % to 25 wt. % of said bulking polymer. In an embodiment, the hydrogel comprises from 1 wt. % to 20 wt. % of said bulking polymer. In certain embodiments, the hydrogel comprises from 1 wt. % to 15 wt. % of said bulking polymer. In an embodiment, the hydrogel comprises from 1 wt. % to 10 wt. % of said bulking polymer. In some embodiments, the hydrogel comprises from 1 wt. % to 5 wt. % of said bulking polymer. In some embodiments, the hydrogel comprises 5 wt. % of said bulking polymer. In an embodiment, the hydrogel comprises 10 wt. % of said bulking polymer. In some embodiments, the hydrogel comprises 15 wt. % of said bulking polymer. In certain embodiments, the hydrogel comprises 20 wt. % of said bulking polymer. In an embodiment, the hydrogel comprises 25 wt. % of said bulking polymer. In an embodiment, the hydrogel comprises 30 wt. % of said bulking polymer. In some embodiments, the hydrogel comprises 35 wt. % of said bulking polymer. In an embodiment, the hydrogel comprises 40 wt. % of said bulking polymer. In various embodiments, the hydrogel comprises 45 wt. % of said bulking polymer. In an embodiment, the hydrogel comprises 50 wt. % of said bulking polymer. In each of the aforementioned embodiments, the bulking polymer is selected from one or more of the above-listed bulking polymers. In a particular embodiment said bulking polymer is gelatin. In some embodiments said bulking polymer is a combination of gelatin and a polymer selected from the above-listed polymers. In some embodiments, said bulking polymer produces a bulking effect and adds rigidity to a hydrogel in the microneedle array or to a hydrogel in the transdermal patch. In an embodiment said bulking polymer is gelatin. In certain embodiments said hydrogel in the microneedle array or in the hydrogel in the transdermal patch bulking polymer is a combination of gelatin and one or more polymers selected from the above-listed polymers. In a particular embodiment, the polymer that produces a bulking effect and adds rigidity to the hydrogel is gelatin. In some embodiments, the sustained delivery hydrogel comprises 5 wt % minoxidil, 5 wt % HA-aldehyde and 30 wt. % gelatin. In particular embodiments, the hydrogel comprises the aldehyde-modified polymer and the minoxidil in a 1:1 ratio. In one embodiment of the sustained delivery hydrogel, the polymer alternatively comprises (i) a secondary Schiff base cross linking mechanism, wherein the cross linking mechanism does not comprise minoxidil or (ii) an orthogonal cross linking selected from the group consisting of Michael additions, Diels-Alder reactions, and azide-alkyne cycloaddition. In an embodiment of the sustained delivery hydrogel, the polymer is a methacrylated polymer.

In another aspect, provided herein is a sustained delivery hydrogel comprising a crosslinked minoxidil-aldehyde-modified hyaluronic acid derivative for treatment of hair loss. In some embodiments, the sustained delivery hydrogel is formulated for transdermal administration or local administration, wherein the transdermal administration comprises subcutaneous injection, microneedle array injection or application of a transdermal patch comprising the hydrogel; wherein the local administration comprises topical application of an ointment, gel, nanoparticles or liposomes comprising the hydrogel. In an embodiment, the hair loss is androgenetic alopecia, alopecia areata, frontal fibrosing alopecia, diffuse alopecia, scarring alopecia or wherein the alopecia areata is universal alopecia (alopecia universalis). In certain embodiments, the hair loss is male or female pattern alopecia. In various embodiments of the sustained delivery hydrogel, the hydrogel comprises the aldehyde-modified hyaluronic acid derivative at a weight percent (wt. %) of from 1 wt. % to 10 wt. % and the minoxidil at wt. % of from 0.1 wt. % to 5 wt. %. In an embodiment of the sustained delivery hydrogel, the hydrogel comprises the aldehyde-modified hyaluronic acid derivative and the minoxidil in a 1:1 ratio. In one embodiment of the sustained delivery hydrogel, the hydrogel polymer alternatively comprises (i) a secondary Schiff base cross linking mechanism, wherein the cross linking mechanism does not comprise minoxidil or (ii) an orthogonal cross linking selected from the group consisting of Michael additions, Diels-Alder reactions, and azide-alkyne cycloaddition. In an embodiment of the sustained delivery hydrogel, the polymer is a methacrylated polymer.

In still another aspect, provided herein is a sustained delivery hydrogel comprising a crosslinked minoxidil-aldehyde-modified hyaluronic acid derivative for treatment of hair loss having chemical structure I:

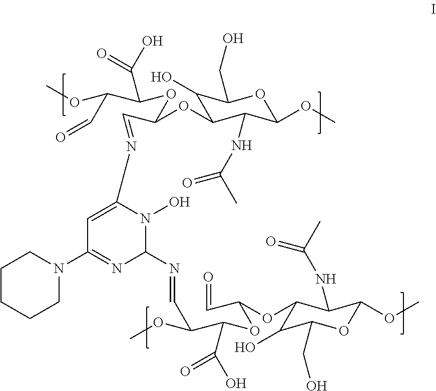

In an embodiment, the sustained delivery hydrogel is formulated for transdermal administration or local administration, wherein the transdermal administration comprises subcutaneous injection, microneedle array injection or application of a transdermal patch comprising the hydrogel; and wherein the local administration comprises topical application of an ointment, gel, nanoparticles or liposomes comprising the hydrogel. In an embodiment, the hair loss is androgenetic alopecia, alopecia areata, frontal fibrosing alopecia, diffuse alopecia, scarring alopecia or wherein the alopecia areata is universal alopecia (alopecia universalis). In certain embodiments, the hair loss is male or female pattern alopecia. In some embodiments, the sustained delivery hydrogel comprises the aldehyde-modified hyaluronic acid derivative at a weight percent (wt. %) of from 1 wt. % to 10 wt. % and the minoxidil at wt. % of from 0.1 wt. % to 5 wt. %. In various embodiments of the sustained delivery hydrogel, the hydrogel comprises the aldehyde-modified hyaluronic acid derivative and the minoxidil in a 1:1 ratio. In one embodiment of the sustained delivery hydrogel, the polymer alternatively comprises (i) a secondary Schiff base cross linking mechanism, wherein the cross linking mechanism does not comprise minoxidil or (ii) an orthogonal cross linking selected from the group consisting of Michael additions, Diels-Alder reactions, and azide-alkyne cycloaddition. In an embodiment of the sustained delivery hydrogel, the polymer is a methacrylated polymer.

In one aspect, provided herein is a method for producing a sustained delivery hydrogel comprising a crosslinked minoxidil-aldehyde-modified polymer, the method comprising: (a) preparing an aldehyde-modified polymer; (b) dissolving the aldehyde-modified polymer in a minoxidil solution comprising phosphate buffered saline; and (c) mixing the minoxidil solution comprising the dissolved aldehyde-modified polymer to react aldehydes of the aldehyde-modified polymer and primary amines of the minoxidil, thereby forming the hydrogel. In an embodiment, the sustained delivery hydrogel comprising the crosslinked minoxidil-aldehyde-modified polymer is formed over 24 to 48 hours. In some embodiments of the provided method, the hydrogel comprises (i) the aldehyde-modified polymer at a weight percent (wt. %) of from 1 wt. % to 10 wt. % and (ii) the minoxidil at wt. % of from 0.1 wt, % to 5 wt. %. In certain embodiments, the polymer is selected from the group consisting of collagen, elastin, thrombin, fibronectin, starches, poly(amino acid), poly(propylene fumarate), gelatin, alginate, pectin, fibrin, oxidized cellulose, chitin, chitosan, tropoelastin, hyaluronic acid, polyethylene, polyethylene terephthalate, poly(tetrafluoroethylene), polycarbonate, polypropylene and poly(vinyl alcohol), ribonucleic acids, deoxyribonucleic acids, polypeptides, proteins, polysaccharides, and polynucleotides. In an embodiment, the hydrogel comprises the aldehyde-modified polymer and the minoxidil in a 1:1 ratio. In some embodiments, the hydrogel is formulated for transdermal administration or local administration, wherein the transdermal administration comprises subcutaneous injection, microneedle array injection or application of a transdermal patch comprising the hydrogel; and wherein the local administration comprises topical application of an ointment, gel, nanoparticles or liposomes comprising the hydrogel.

In another aspect, provided herein is a method for producing a sustained delivery hydrogel comprising a crosslinked minoxidil-aldehyde-modified hyaluronic acid derivative, the method comprising: (a) preparing an aldehyde-modified hyaluronic acid derivative; (b) dissolving the aldehyde-modified hyaluronic acid derivative in a minoxidil solution comprising phosphate buffered saline; and (c) mixing the minoxidil solution comprising the dissolved aldehyde-modified polymer to react aldehydes of the aldehyde-modified polymer and primary amines of the minoxidil, thereby forming the hydrogel. In an embodiment of the method, the sustained delivery hydrogel comprising the crosslinked minoxidil-aldehyde-modified hyaluronic acid derivative is formed over 24 to 48 hours. In some embodiments of the herein provided method, the sustained delivery hydrogel comprises (i) the aldehyde-modified hyaluronic acid derivative at a weight percent (wt. %) of from 1 wt. % to 10 wt. % and (ii) the minoxidil at wt. % of from 0.1 wt. % to 5 wt. %. In an embodiment, the hydrogel comprises the minoxidil-aldehyde-modified hyaluronic acid derivative and the minoxidil in a 1:1 ratio. In some embodiments, the hydrogel is formulated for transdermal administration or local administration, wherein the transdermal administration comprises subcutaneous injection, microneedle array injection or application of a transdermal patch comprising the hydrogel; and wherein the local administration comprises topical application of an ointment, gel, nanoparticles or liposomes comprising the hydrogel. In a particular embodiment, the sustained delivery hydrogel has chemical structure I:

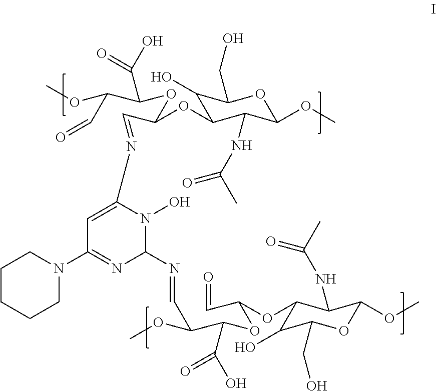

In still another aspect, provided herein is a method for producing a sustained delivery hydrogel comprising a crosslinked minoxidil-aldehyde-modified hyaluronic acid derivative, the method comprising: (a) preparing an aldehyde-modified hyaluronic acid derivative; (b) dissolving the aldehyde-modified hyaluronic acid derivative in a minoxidil solution comprising phosphate buffered saline; and (c) mixing the minoxidil solution comprising the dissolved aldehyde-modified polymer to react aldehydes of the aldehyde-modified polymer and primary amines of the minoxidil, thereby forming the hydrogel. In an embodiment of the herein provided method, administration of the hydrogel induces anagen phase of hair growth, prolongs the anagen phase, increases hair follicle size, reverses, prevents or inhibits miniaturization of the hair follicle, and combinations thereof. In an embodiment, the hair loss is androgenetic alopecia, alopecia areata, frontal fibrosing alopecia, diffuse alopecia, scarring alopecia or wherein the alopecia areata is universal alopecia (alopecia universalis). In certain embodiments, the hair loss is male or female pattern alopecia. In certain embodiments, the hair loss is scalp or beard hair loss. In a particular embodiment of the herein provided method, the hydrogel comprises (i) the aldehyde-modified polymer at a weight percent (wt. %) of from 1 wt. % to 10 wt. % and (ii) the minoxidil at wt. % of from 0.1 wt. % to 5 wt. %. In various embodiments, the polymer is selected from the group consisting of collagen, elastin, thrombin, fibronectin, starches, poly(amino acid), poly(propylene fumarate), gelatin, alginate, pectin, fibrin, oxidized cellulose, chitin, chitosan, tropoelastin, hyaluronic acid, polyethylene, polyethylene terephthalate, poly(tetrafluoroethylene), polycarbonate, polypropylene and poly(vinyl alcohol), ribonucleic acids, deoxyribonucleic acids, polypeptides, proteins, polysaccharides, and polynucleotides. In a particular embodiment of the herein provided methods, the hydrogel comprises the aldehyde-modified polymer and the minoxidil in a 1:1 ratio.

In another aspect, provided herein is a method for treating hair loss in a subject in need thereof, the method comprising administering a sustained delivery hydrogel comprising a crosslinked minoxidil-aldehyde-modified hyaluronic acid derivative to an area of hair loss of the subject, wherein administration comprises: (a) transdermal administration comprising subcutaneous injection, microneedle array injection or application of a transdermal patch comprising the hydrogel; (b) local administration comprising topical application of an ointment, gel, nanoparticles or liposomes comprising the hydrogel; or (c) a combination of (i) skin needling, and (ii) the local administration of (b). In an embodiment of the herein provided method, administration of the hydrogel induces anagen phase of hair growth, prolongs the anagen phase, increases hair follicle size, reverses, prevents or inhibits miniaturization of the hair follicle, and combinations thereof. In an embodiment, the hair loss is androgenetic alopecia, alopecia areata, frontal fibrosing alopecia, diffuse alopecia, scarring alopecia or wherein the alopecia areata is universal alopecia (alopecia universalis). In certain embodiments, the hair loss is male or female pattern alopecia. In some embodiments, the hydrogel comprises (i) the aldehyde-modified hyaluronic acid derivative at a weight percent (wt. %) of from 1 wt. % to 10 wt. % and (ii) the minoxidil at wt. % of from 0.1 wt. % to 5 wt. %. In certain embodiments, the hydrogel comprises the polymer and the minoxidil in a 1:1 ratio. In a particular embodiment, the sustained delivery hydrogel has chemical structure I:

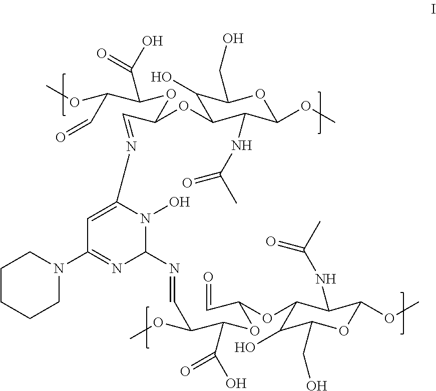

In one aspect, provided herein is a method for prolonging anagen in a subject having hair loss, the method comprising administering a sustained delivery hydrogel comprising a crosslinked minoxidil-aldehyde-modified hyaluronic acid derivative to an area of hair loss of the subject, wherein administration comprises: (a) transdermal administration comprising subcutaneous injection, microneedle array injection or application of a transdermal patch comprising the hydrogel; (b) local administration comprising topical application of an ointment, gel, nanoparticles or liposomes comprising the hydrogel; or (c) a combination of (i) skin needling, and (ii) the local administration of (b). In one embodiment, administration of the hydrogel prolongs the anagen phase by 2 to 4 months. In an embodiment of the herein provided method, administration of the hydrogel induces anagen phase of hair growth, prolongs the anagen phase, increases hair follicle size, reverses, prevents or inhibits miniaturization of the hair follicle, and combinations thereof. In an embodiment, the hair loss is androgenetic alopecia, alopecia areata, frontal fibrosing alopecia, diffuse alopecia, scarring alopecia or wherein the alopecia areata is universal alopecia (alopecia universalis). In certain embodiments, the hair loss is male or female pattern alopecia. In an embodiment, the hair loss is scalp or beard hair loss. In an embodiment of the herein provided method, the hydrogel comprises (i) the aldehyde-modified hyaluronic acid derivative at a weight percent (wt. %) of from 1 wt. % to 10 wt. % and (ii) the minoxidil at wt. % of from 0.1 wt. % to 5 wt. %. In some embodiments, the hydrogel comprises the polymer and the minoxidil in a 1:1 ratio. In a particular embodiment of the herein provided method, the sustained delivery hydrogel has chemical structure I:

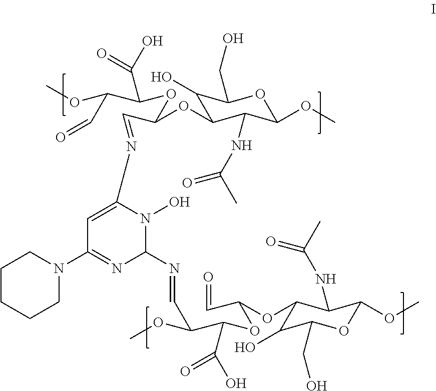

In another aspect, provided herein is a method for prolonging anagen in a subject having hair loss, the method comprising administering a sustained delivery hydrogel comprising a crosslinked minoxidil-aldehyde-modified polymer to an area of hair loss of the subject, wherein administration comprises: (a) transdermal administration comprising subcutaneous injection, microncedle array injection or application of a transdermal patch comprising the hydrogel; (b) local administration comprising topical application of an ointment, gel, nanoparticles or liposomes comprising the hydrogel; or (c) a combination of (i) skin needling, and (ii) the local administration of (b). In certain embodiments, administration of the hydrogel prolongs the anagen phase by 2 to 4 months. In an embodiment of the herein provided method, administration of the hydrogel induces anagen phase of hair growth, prolongs the anagen phase, increases hair follicle size, reverses, prevents or inhibits miniaturization of the hair follicle, and combinations thereof. In an embodiment, the hair loss is androgenetic alopecia, alopecia areata, frontal fibrosing alopecia, diffuse alopecia, scarring alopecia or wherein the alopecia areata is universal alopecia (alopecia universalis). In certain embodiments, the hair loss is male or female pattern alopecia. In various embodiments, the hair loss is scalp or beard hair loss. In a particular embodiment, the hydrogel comprises (i) the aldehyde-modified polymer at a weight percent (wt. %) of from 1 wt. % to 10 wt. % and (ii) the minoxidil at wt. % of from 0.1 wt. % to 5 wt. %. In some embodiments, the polymer is selected from the group consisting of collagen, elastin, thrombin, fibronectin, starches, poly(amino acid), poly(propylene fumarate), gelatin, alginate, pectin, fibrin, oxidized cellulose, chitin, chitosan, tropoelastin, hyaluronic acid, polyethylene, polyethylene terephthalate, poly(tetrafluoroethylene), polycarbonate, polypropylene and poly(vinyl alcohol), ribonucleic acids, deoxyribonucleic acids, polypeptides, proteins, polysaccharides, and polynucleotides. In an embodiment of the herein provided methods for prolonging anagen in the subject, the hydrogel comprises the aldehyde-modified polymer and the minoxidil in a 1:1 ratio.

Administration via the herein described methods of the herein described sustained delivery hydrogels comprising minoxidil crosslinked to an aldehyde polymer or application of the herein described hydrogels comprising minoxidil crosslinked to an aldehyde polymer and a polymer that provides bulking and rigidity to the hydrogel to an area of hair loss of a subject induces hair growth in three weeks to six months. In an embodiment, hair growth is induced by the administration of said hydrogels in three to six weeks. In some embodiments, hair growth is induced by the administration of said hydrogels in four to six weeks. In an embodiment, hair growth is induced by the administration of said hydrogels in five to six weeks. In certain embodiments, hair growth is induced by the administration of said hydrogels in three weeks. In some embodiments, hair growth is induced by the administration of said hydrogels in four weeks. In an embodiment, hair growth is induced by the administration of said hydrogels in five weeks. In various embodiments, hair growth is induced by the administration of said hydrogels in six weeks. In an embodiment, hair growth is induced by the administration of said hydrogels in six weeks to two months. In certain embodiments, hair growth is induced by the administration of said hydrogels in six weeks to three months. In some embodiments, hair growth is induced by the administration of said hydrogels in six weeks to four months. In an embodiments, hair growth is induced by the administration of said hydrogels in six weeks to five months. In some embodiments, hair growth is induced by the administration of said hydrogels in six weeks to six months. In certain embodiments, hair growth is induced by the administration of said hydrogels in one to six months. In an a embodiment, hair growth is induced by the administration of said hydrogels in two to six months. In certain embodiments, hair growth is induced by the administration of said hydrogels in three to six months. In some embodiments, hair growth is induced by the administration of said hydrogels in four to six months. In various embodiments, hair growth is induced by the administration of said hydrogels in five to six months. In some embodiments, hair growth is induced by the administration of said hydrogels in six months.

The following examples are presented in order to more fully illustrate the preferred embodiments of the invention. They should in no way be construed, however, as limiting the broad scope of the invention.

### EXAMPLES

### Example 1: Preparation of a Sustained Delivery Hydrogel Comprising a Crosslinked Minoxidil-Aldehyde-Modified Polymer

Hyaluronic acid (HA) (400 kDa, 200 mg) and sodium periodate (103 mg, 1:1 periodate/HA molar ratio) was dissolved in 20 mL dH2O. The reaction was stirred for two hours in the dark and subsequently quenched in 2 mL ethylene glycol. Then, the solution was dialyzed for five days against dH2O (14000 MWCO) and lyophilized. The prepared aldehyde-modified HA (FIG. 1A) was stored under nitrogen at −20° C. until use.

To form hydrogels comprising a crosslinked minoxidil-aldehyde-modified hyaluronic acid derivative hydrogels, aldehyde-modified HA is dissolved in minoxidil solutions in phosphate buffered saline, wherein the aldehyde-modified HA and minoxidil were assembled at the appropriate weight percentages and mixed (FIG. 1B). For example, the aldehyde-modified polymer (e.g., the HA) may be at a weight percent (wt. %) of from 1 wt. % to 10 wt. % and the minoxidil at a wt. % of from 0.1 wt. % to 5 wt. %.

Hydrogels slowly form over 24-48 hours before they can be loaded into syringes; alternatively, the polymer can be directly implanted into a syringe and dissolved in situ in the minoxidil solution. The above-described reactions occur at a room temperature of from 20° C. to 25° C. to 37 C.

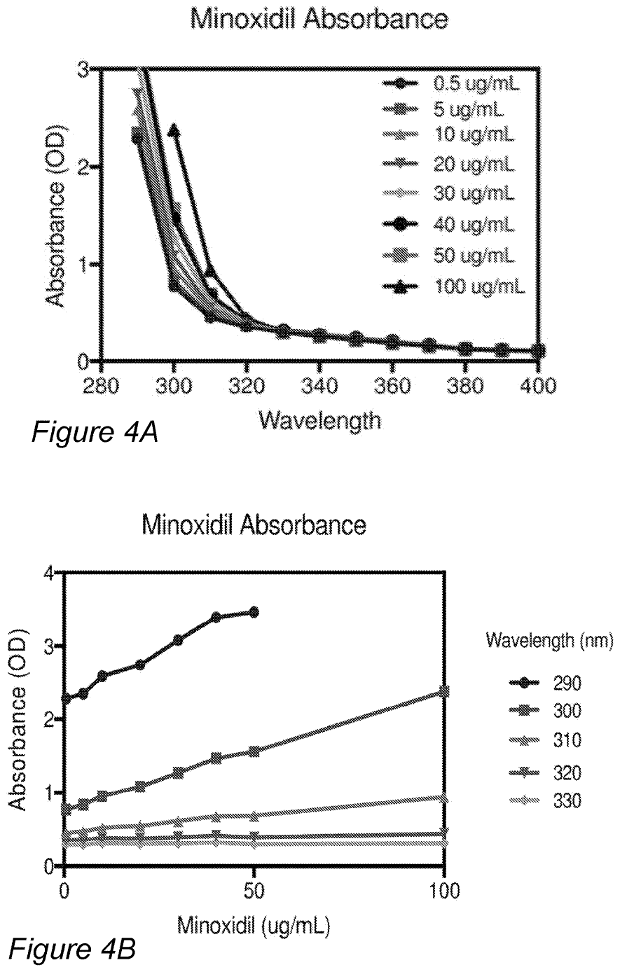

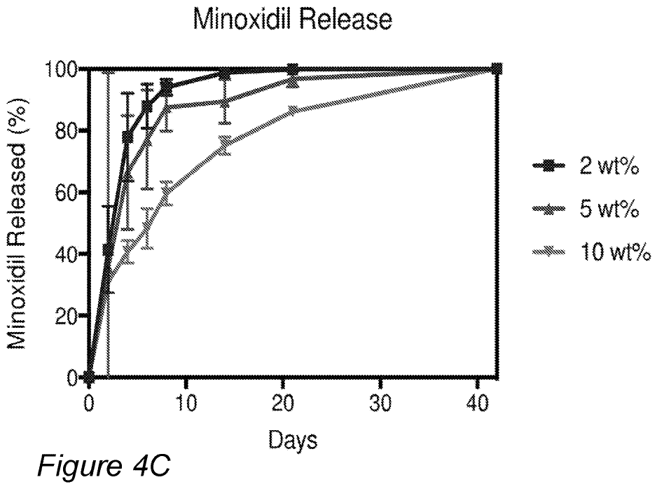

Characterization of the minoxidil-aldehyde-modified hyaluronic acid derivative hydrogel showed that the storage modulus was approximately mean 339.3 SD 32.2 for a formulation of 2 wt. % aldehyde-modified HA. The prepared hydrogels are soft materials. A prepared crosslinked minoxidil-aldehyde-modified hyaluronic acid derivative hydrogel is shown in FIG. 2. The absorbance of various minoxidil concentrations is shown in FIGS. 4A-4B. Minoxidil release was determined by UV spectroscopy, as shown in FIG. 4C.

In addition to HA, other polymers that may be used for aldehyde modification and crosslinking with minoxidil include collagen, elastin, thrombin, fibronectin, starches, poly(amino acid), poly(propylene fumarate), gelatin, alginate, pectin, fibrin, oxidized cellulose, chitin, chitosan, tropoelastin, polyethylene, polyethylene terephthalate, poly(tetrafluoroethylene), polycarbonate, polypropylene and poly(vinyl alcohol), ribonucleic acids, deoxyribonucleic acids, polypeptides, proteins, polysaccharides, polynucleotides

### Example 2: Anagen Induction by Sustained Delivery Hydrogel Comprising a Crosslinked Minoxidil-Aldehyde-Modified Hyaluronic Acid Derivative

Nair™ hair remover was applied to dorsal skin of telogen phase eight week old mice. The mice were subsequently injected with either an aldehyde-modified hyaluronic acid alone (control) (FIG. 3A), hydrogels crosslinked with minoxidil (FIG. 3B) or minoxidil sulfate at varying concentrations (FIGS. 3C-3D). Two five microliter injections of hydrogel were administered superiorly (red boxes). A 30 gauge (0.312 mm) needle was used for the injections. Local anagen induction was observed in multiple mice after 12 days (red arrows), which was seen only in the minoxidil hydrogel treatment groups.

### Example 3: Anagen Induction by Microneedle Application of Sustained Delivery Hydrogel Comprising a Crosslinked Minoxidil-Aldehyde-Modified Hyaluronic Acid Derivative

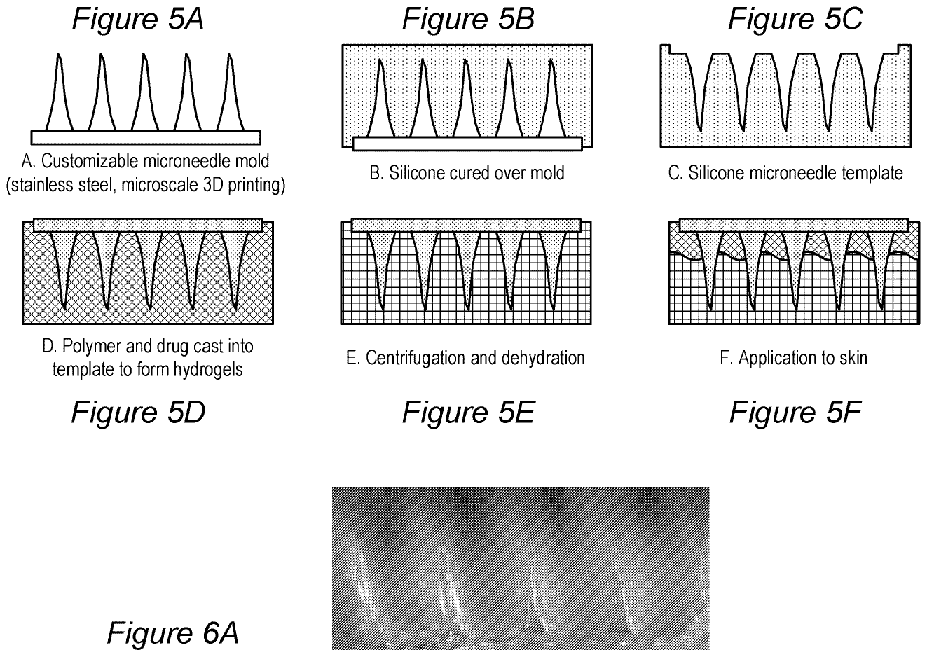

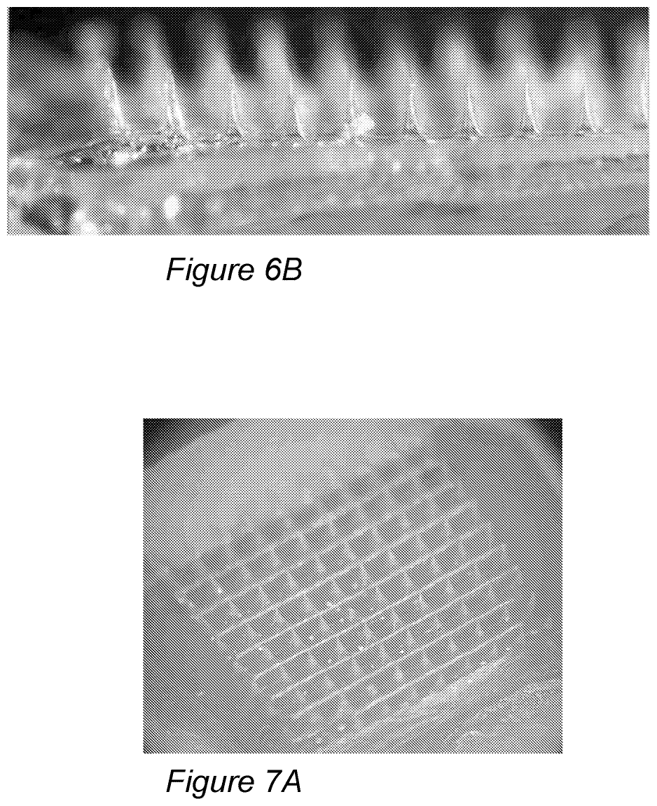

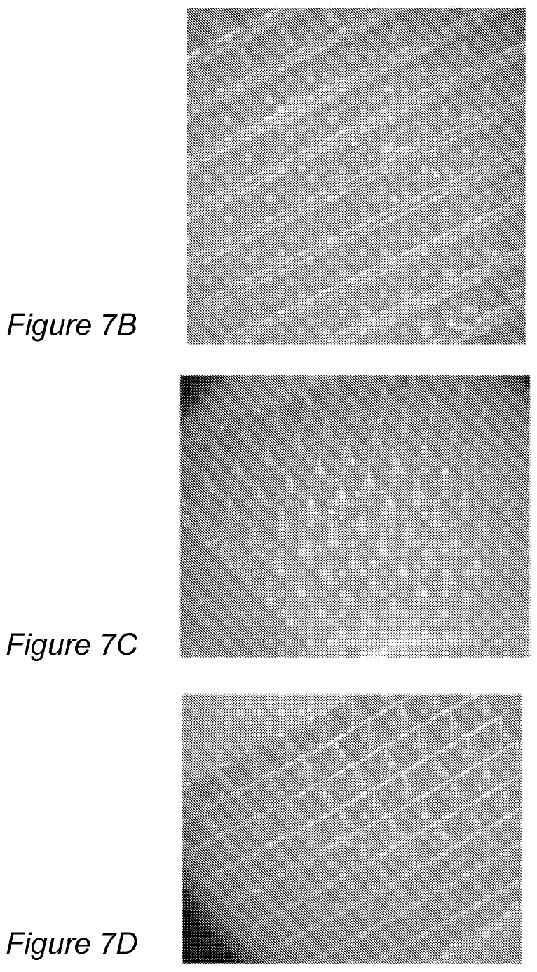

Microneedle templates having a 600 μm height and a 200 μm base by 500 μm base were formed through casting vulcanizing silicone over a stainless steel master mold. Hydrogel microneedle patches were assembled by mixing and dissolving polymers to weight percentages of 5 wt. % minoxidil, 5 wt. % HA-aldehyde (minoxidil-aldehyde-modified hyaluronic acid derivative hydrogel), and 30 wt. % gelatin, pipetting 100 μL directly into molds (FIGS. 5A-5D), centrifugation (FIG. 5C), followed by desiccation under vacuum for 48 hours (FIG. 5C). Molds were removed with Scotch tape and subsequently transferred underneath a transparent medical dressing (Tegaderm™) before application to mice in telogen (FIG. 5F). The microneedle patches (10×10, 1 cm²) are shown in FIGS. 6A-6B with light microscopy. The microneedle patches (10×10, 1 cm²) are also shown with red dye for contrast in FIGS. 7A-7D.

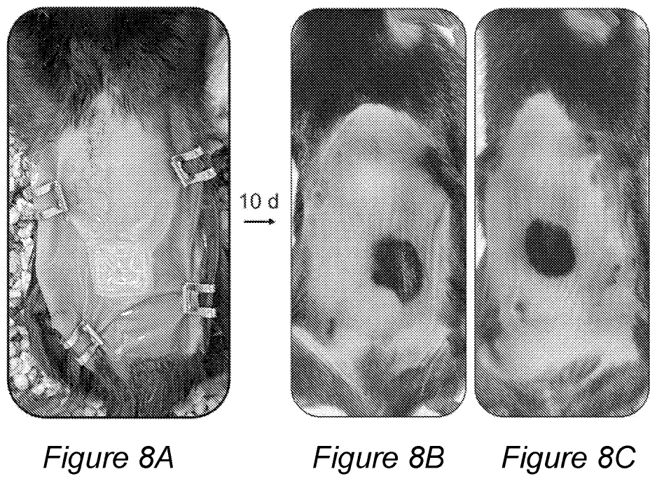

For anagen induction experiments, B6 female mice in telogen had dorsal hair clipped at 8 weeks. Microneedle patches comprising the minoxidil, crosslinked HA-aldehyde and gelatin hydrogels were applied manually to the skin followed by application of Tegaderm™ dressing and clipping, as shown in FIG. 8A. Dressing was removed at 10 days. The application of the microneedle patch comprising the minoxidil sustained delivery hydrogel (a crosslinked minoxidil-aldehyde-modified hyaluronic acid derivative) induced anagen after 10 days, as shown in FIGS. 8B-8C. FIGS. 8B-8C show hair growth, including at the black spot, where the hair follicles are in anagen that is induced by the microneedle patch comprising the minoxidil, HA-aldehyde and gelatin hydrogel.

Having described certain embodiments of the invention with reference to the accompanying drawings, it is to be understood that the invention is not limited to the precise embodiments, and that various changes and modifications may be effected therein by those skilled in the art without departing from the scope or spirit of the invention as defined in the appended claims.

1. A sustained delivery hydrogel comprising a crosslinked minoxidil-aldehyde-modified polymer for treatment of hair loss.

2. The sustained delivery hydrogel of claim 1 formulated for transdermal administration or local administration, wherein the transdermal administration comprises subcutaneous injection, microneedle array injection or application of a transdermal patch comprising the hydrogel; wherein the local administration comprises topical application of an ointment, gel, nanoparticles or liposomes comprising the hydrogel.

3. The sustained delivery hydrogel of claim 1, wherein the hair loss is androgenetic alopecia, alopecia areata, frontal fibrosing alopecia, diffuse alopecia, scarring alopecia or wherein the alopecia areata is alopecia areata of the universalis type.

4. The sustained delivery hydrogel of claim 1, wherein the hair loss is male or female pattern alopecia.

5. The sustained delivery hydrogel of claim 1, wherein the hydrogel comprises the aldehyde-modified polymer at a weight percent (wt. %) of from 1 wt. % to 10 wt. % and the minoxidil at wt. % of from 0.1 wt. % to 5 wt. %.

6. The sustained delivery hydrogel of claim 1, wherein the polymer is selected from the group consisting of collagen, elastin, thrombin, fibronectin, starches, poly(amino acid), poly(propylene fumarate), gelatin, alginate, pectin, fibrin, oxidized cellulose, chitin, chitosan, tropoelastin, hyaluronic acid, polyethylene, polyethylene terephthalate, poly(tetrafluoroethylene), polycarbonate, polypropylene and poly(vinyl alcohol), ribonucleic acids, deoxyribonucleic acids, polypeptides, proteins, polysaccharides, and polynucleotides.

7. The sustained delivery hydrogel of claim 6, wherein the polymer alternatively comprises (i) a secondary Schiff base cross linking mechanism, wherein the cross linking mechanism does not comprise minoxidil or (ii) an orthogonal cross linking selected from the group consisting of Michael additions, Diels-Alder reactions, and azide-alkyne cycloaddition.

8. The sustained delivery hydrogel of claim 6, wherein the polymer is a methacrylated polymer.

9. The sustained delivery hydrogel of claim 1, wherein the hydrogel comprises the aldehyde-modified polymer and the minoxidil in a 1:1 ratio.

10. A sustained delivery hydrogel comprising a crosslinked minoxidil-aldehyde-modified hyaluronic acid derivative for treatment of hair loss.

11. The sustained delivery hydrogel of claim 10 formulated for transdermal administration or local administration, wherein the transdermal administration comprises subcutaneous injection, microneedle array injection or application of a transdermal patch comprising the hydrogel; wherein the local administration comprises topical application of an ointment, gel, nanoparticles or liposomes comprising the hydrogel.

12. The sustained delivery hydrogel of claim 10, wherein the hair loss is androgenetic alopecia, alopecia areata, frontal fibrosing alopecia, diffuse alopecia, scarring alopecia or wherein the alopecia areata is alopecia areata of the universalis type.

13. The sustained delivery hydrogel of claim 10, wherein the hair loss is male or female alopecia.

14. The sustained delivery hydrogel of claim 10, wherein the hydrogel comprises the aldehyde-modified hyaluronic acid derivative at a weight percent (wt. %) of from 1 wt. % to 10 wt. % and the minoxidil at wt. % of from 0.1 wt. % to 5 wt. %.

15. The sustained delivery hydrogel of claim 10, wherein the hydrogel polymer alternatively comprises (i) a secondary Schiff base cross linking mechanism, wherein the cross linking mechanism does not comprise minoxidil or (ii) an orthogonal cross linking selected from the group consisting of Michael additions, Diels-Alder reactions, and azide-alkyne cycloaddition.

16. The sustained delivery hydrogel of claim 15, wherein the polymer is a methacrylated polymer.

17. The sustained delivery hydrogel of claim 10, wherein the hydrogel comprises the aldehyde-modified hyaluronic acid derivative and the minoxidil in a 1:1 ratio.

18. A sustained delivery hydrogel comprising a crosslinked minoxidil-aldehyde-modified hyaluronic acid derivative for treatment of hair loss having chemical structure I:

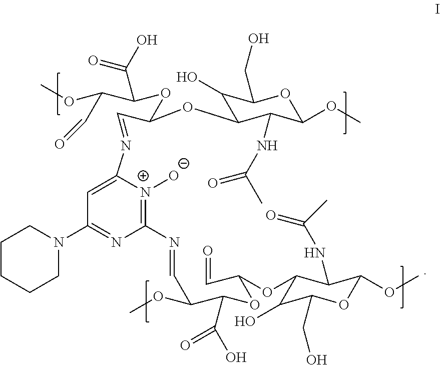

19. The sustained delivery hydrogel of claim 18 formulated for transdermal administration or local administration, wherein the transdermal administration comprises subcutaneous injection, microneedle array injection or application of a transdermal patch comprising the hydrogel; and wherein the local administration comprises topical application of an ointment, gel, nanoparticles or liposomes comprising the hydrogel.

20. The sustained delivery hydrogel of claim 18, wherein the hair loss is androgenetic alopecia, areata alopecia, frontal fibrosing alopecia, diffuse alopecia, scarring alopecia or wherein the areata alopecia is universal alopecia.

21. The sustained delivery hydrogel of claim 18, wherein the hair loss is male or female alopecia.

22. The sustained delivery hydrogel of claim 18, wherein the hydrogel comprises the aldehyde-modified hyaluronic acid derivative at a weight percent (wt. %) of from 1 wt. % to 10 wt. % and the minoxidil at wt. % of from 0.1 wt. % to 5 wt. %.

23. The sustained delivery hydrogel of claim 18, wherein the hydrogel comprises the aldehyde-modified hyaluronic acid derivative and the minoxidil in a 1:1 ratio.

24. The sustained delivery hydrogel of claim 23, wherein the hydrogel comprises the aldehyde-modified hyaluronic acid derivative and the minoxidil in a 1:1 ratio.

25. A method for producing a sustained delivery hydrogel comprising a crosslinked minoxidil-aldehyde-modified polymer, the method comprising:
(a) preparing an aldehyde-modified polymer;
(b) dissolving the aldehyde-modified polymer in a minoxidil solution comprising phosphate buffered saline; and
(c) mixing the minoxidil solution comprising the dissolved aldehyde-modified polymer to react aldehydes of the aldehyde-modified polymer and primary amines of the minoxidil, thereby forming the hydrogel.

26. The method of claim 25, wherein the sustained delivery hydrogel comprising the crosslinked minoxidil-aldehyde-modified polymer is formed over 24 to 48 hours.

27. The method of claim 25, wherein the hydrogel comprises (i) the aldehyde-modified polymer at a weight percent (wt. %) of from 1 wt. % to 10 wt. % and (ii) the minoxidil at a wt. % of from 0.1 wt. % to 5 wt. %.

28. The method of claim 25, wherein the polymer is selected from the group consisting of collagen, elastin, thrombin, fibronectin, starches, poly(amino acid), poly(propylene fumarate), gelatin, alginate, pectin, fibrin, oxidized cellulose, chitin, chitosan, tropoelastin, hyaluronic acid, polyethylene, polyethylene terephthalate, poly(tetrafluoroethylene), polycarbonate, polypropylene and poly(vinyl alcohol), ribonucleic acids, deoxyribonucleic acids, polypeptides, proteins, polysaccharides, and polynucleotides.

29. The method of claim 25, wherein the hydrogel comprises the aldehyde-modified polymer and the minoxidil in a 1:1 ratio.

30. The method of claim 25, wherein the hydrogel is formulated for transdermal administration or local administration, wherein the transdermal administration comprises subcutaneous injection, microneedle array injection or application of a transdermal patch comprising the hydrogel; and wherein the local administration comprises topical application of an ointment, gel, nanoparticles or liposomes comprising the hydrogel.

31. A method for producing a sustained delivery hydrogel comprising a crosslinked minoxidil-aldehyde-modified hyaluronic acid derivative, the method comprising:
(a) preparing an aldehyde-modified hyaluronic acid derivative;
(b) dissolving the aldehyde-modified hyaluronic acid derivative in a minoxidil solution comprising phosphate buffered saline; and
(c) mixing the minoxidil solution comprising the dissolved aldehyde-modified polymer to react aldehydes of the aldehyde-modified polymer and primary amines of the minoxidil, thereby forming the hydrogel.

32. The method of claim 31, wherein the sustained delivery hydrogel comprising the crosslinked minoxidil-aldehyde-modified hyaluronic acid derivative is formed over 24 to 48 hours.

33. The method of claim 31, wherein the sustained delivery hydrogel comprises (i) the aldehyde-modified hyaluronic acid derivative at a weight percent (wt. %) of from 1 wt. % to 10 wt. % and (ii) the minoxidil at a wt. % of from 0.1 wt. % to 5 wt. %.

34. The method of claim 31, wherein the hydrogel comprises the minoxidil-aldehyde-modified hyaluronic acid derivative and the minoxidil in a 1:1 ratio.

35. The method of claim 31, wherein the hydrogel is formulated for transdermal administration or local administration, wherein the transdermal administration comprises subcutaneous injection, microneedle array injection or application of a transdermal patch comprising the hydrogel; and wherein the local administration comprises topical application of an ointment, gel, nanoparticles or liposomes comprising the hydrogel.

36. The method of claim 31, wherein the sustained delivery hydrogel has chemical structure I:

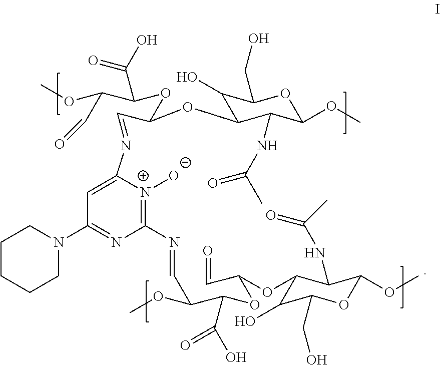

37. A method for treating hair loss in a subject in need thereof, the method comprising administering a sustained delivery hydrogel comprising a crosslinked minoxidil-aldehyde-modified polymer to an area of hair loss of the subject,
wherein administration comprises:
(a) transdermal administration comprising subcutaneous injection, microneedle array injection or application of a transdermal patch comprising the hydrogel;
(b) local administration comprising topical application of an ointment, gel, nanoparticles or liposomes comprising the hydrogel; or
(c) a combination of (i) skin needling, and (ii) the local administration of (b).

38. The method of claim 37, wherein administration of the hydrogel induces anagen phase of hair growth, prolongs the anagen phase, increases hair follicle size, reverses, prevents or inhibits miniaturization of the hair follicle, and combinations thereof.

39. The method of claim 37, wherein the hair loss is androgenetic alopecia, alopecia areata, frontal fibrosing alopecia, diffuse alopecia, scarring alopecia or wherein the alopecia areata is of the universalis type.

40. The method of claim 37, wherein the hair loss is male or female pattern alopecia.

41. The method of claim 37, wherein the hair loss is scalp or beard hair loss.

42. The method of claim 37, wherein the hydrogel comprises (i) the aldehyde-modified polymer at a weight percent (wt. %) of from 1 wt. % to 10 wt. % and (ii) the minoxidil at a wt. % of from 0.1 wt. % to 5 wt. %.

43. The method of claim 37, wherein the polymer is selected from the group consisting of collagen, elastin, thrombin, fibronectin, starches, poly(amino acid), poly(propylene fumarate), gelatin, alginate, pectin, fibrin, oxidized cellulose, chitin, chitosan, tropoelastin, hyaluronic acid, polyethylene, polyethylene terephthalate, poly(tetrafluoroethylene), polycarbonate, polypropylene and poly(vinyl alcohol), ribonucleic acids, deoxyribonucleic acids, polypeptides, proteins, polysaccharides, and polynucleotides.

44. The method of claim 37, wherein the hydrogel comprises the aldehyde-modified polymer and the minoxidil in a 1:1 ratio.

45. A method for treating hair loss in a subject in need thereof, the method comprising administering a sustained delivery hydrogel comprising a crosslinked
minoxidil-aldehyde-modified hyaluronic acid derivative to an area of hair loss of the subject,
wherein administration comprises:
(c) transdermal administration comprising subcutaneous injection, microneedle array injection or application of a transdermal patch comprising the hydrogel;
(d) local administration comprising topical application of an ointment, gel, nanoparticles or liposomes comprising the hydrogel; or
(c) a combination of (i) skin needling, and (ii) the local administration of (b).

46. The method of claim 45, wherein administration of the hydrogel induces anagen phase of hair growth, prolongs the anagen phase, increases hair follicle size, reverses, prevents or inhibits miniaturization of the hair follicle, and combinations thereof.

47. The method of claim 45, wherein the hair loss is androgenetic alopecia, alopecia areata, frontal fibrosing alopecia, diffuse alopecia, scarring alopecia or wherein the alopecia areata is of the universalis type.

48. The method of claim 45, wherein the hair loss is male or female pattern alopecia.

49. The method of claim 45, wherein the hair loss is scalp or beard hair loss.

50. The method of claim 45, wherein the hydrogel comprises (i) the aldehyde-modified hyaluronic acid derivative at a weight percent (wt. %) of from 1 wt. % to 10 wt. % and (ii) the minoxidil at a wt. % of from 0.1 wt. % to 5 wt. %.

51. The method of claim 45, wherein the hydrogel comprises the polymer and the minoxidil in a 1:1 ratio.

52. The method of claim 45, wherein the sustained delivery hydrogel has chemical structure I:

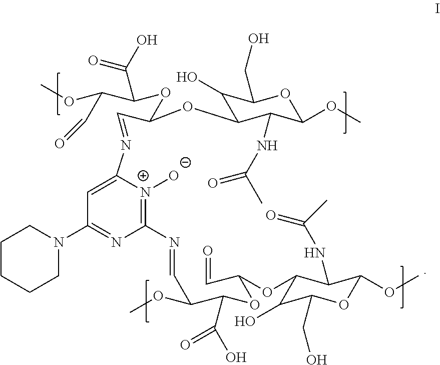

53. A method for prolonging anagen in a subject having hair loss, the method comprising administering a sustained delivery hydrogel comprising a crosslinked
minoxidil-aldehyde-modified hyaluronic acid derivative to an area of hair loss of the subject,
wherein administration comprises:
(a) transdermal administration comprising subcutaneous injection, microneedle array injection or application of a transdermal patch comprising the hydrogel;
(b) local administration comprising topical application of an ointment, gel, nanoparticles or liposomes comprising the hydrogel; or
(c) a combination of (i) skin needling, and (ii) the local administration of (b).

54. The method of claim 53, wherein administration of the hydrogel prolongs the anagen phase by 2 to 4 months.

55. The method of claim 53, wherein the hair loss androgenetic alopecia, alopecia areata, frontal fibrosing alopecia, diffuse alopecia, scarring alopecia or wherein the alopecia areata is of the universalis type.

56. The method of claim 53, wherein the hair loss is male or female pattern alopecia.

57. The method of claim 53, wherein the hair loss is scalp or beard hair loss.

58. The method of claim 53, wherein the hydrogel comprises (i) the aldehyde-modified hyaluronic acid derivative at a weight percent (wt. %) of from 1 wt. % to 10 wt. % and (ii) the minoxidil at a wt. % of from 0.1 wt. % to 5 wt. %.

59. The method of claim 53, wherein the hydrogel comprises the polymer and the minoxidil in a 1:1 ratio.

60. The method of claim 53, wherein the sustained delivery hydrogel has chemical structure I:

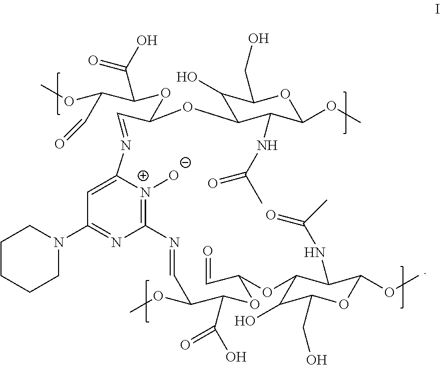

61. A method for prolonging anagen in a subject having hair loss, the method comprising administering a sustained delivery hydrogel comprising a crosslinked minoxidil-aldehyde-modified polymer to an area of hair loss of the subject,
wherein administration comprises:
(a) transdermal administration comprising subcutaneous injection, microneedle array injection or application of a transdermal patch comprising the hydrogel;
(b) local administration comprising topical application of an ointment, gel, nanoparticles or liposomes comprising the hydrogel; or
(c) a combination of (i) skin needling, and (ii) the local administration of (b).

62. The method of claim 61, wherein administration of the hydrogel prolongs the anagen phase by 2 to 4 months.

63. The method of claim 61, wherein the hair loss is androgenetic alopecia, alopecia areata, frontal fibrosing alopecia, diffuse alopecia, scarring alopecia or wherein the alopecia areata is of the universalis type.

64. The method of claim 61, wherein the hair loss is male or female pattern alopecia.

65. The method of claim 61, wherein the hair loss is scalp or beard hair loss.

66. The method of claim 61, wherein the hydrogel comprises (i) the aldehyde-modified polymer at a weight percent (wt. %) of from 1 wt. % to 10 wt. % and (ii) the minoxidil at a wt. % of from 0.1 wt. % to 5 wt. %.

67. The method of claim 61, wherein the polymer is selected from the group consisting of collagen, elastin, thrombin, fibronectin, starches, poly(amino acid), poly(propylene fumarate), gelatin, alginate, pectin, fibrin, oxidized cellulose, chitin, chitosan, tropoelastin, hyaluronic acid, polyethylene, polyethylene terephthalate, poly(tetrafluoroethylene), polycarbonate, polypropylene and poly(vinyl alcohol), ribonucleic acids, deoxyribonucleic acids, polypeptides, proteins, polysaccharides, and polynucleotides.

68. The method of claim 61, wherein the hydrogel comprises the aldehyde-modified polymer and the minoxidil in a 1:1 ratio.

69. A method for methods for reversing, preventing or inhibiting hair follicle miniaturization in a subject having hair loss, comprising administering a sustained delivery hydrogel comprising a crosslinked minoxidil-aldehyde-modified polymer to an area of hair loss of the subject.

70. The method of claim 69, wherein the hair loss is androgenetic alopecia, alopecia areata, frontal fibrosing alopecia, diffuse alopecia, scarring alopecia or wherein the alopecia areata is of the universalis type.

71. The method of claim 69, wherein the hair loss is male or female pattern alopecia.

72. The method of claim 69, wherein the hair loss is scalp or beard hair loss.

73. The method of claim 69, wherein the hydrogel comprises (i) the aldehyde-modified polymer at a weight percent (wt. %) of from 1 wt. % to 10 wt. % and (ii) the minoxidil at a wt. % of from 0.1 wt. % to 5 wt. %.

74. The method of claim 69, wherein the polymer is selected from the group consisting of collagen, elastin, thrombin, fibronectin, starches, poly(amino acid), poly(propylene fumarate), gelatin, alginate, pectin, fibrin, oxidized cellulose, chitin, chitosan, tropoelastin, hyaluronic acid, polyethylene, polyethylene terephthalate, poly(tetrafluoroethylene), polycarbonate, polypropylene and poly(vinyl alcohol), ribonucleic acids, deoxyribonucleic acids, polypeptides, proteins, polysaccharides, and polynucleotides.

75. The method of claim 69, wherein the hydrogel comprises the aldehyde-modified polymer and the minoxidil in a 1:1 ratio.

76. A method for methods for reversing, preventing or inhibiting hair follicle miniaturization in a subject having hair loss, comprising administering a sustained delivery hydrogel comprising a crosslinked minoxidil-aldehyde-modified hyaluronic acid derivative to an area of hair loss of the subject.

77. The method of claim 76, wherein the hair loss androgenetic alopecia, alopecia areata, frontal fibrosing alopecia, diffuse alopecia, scarring alopecia or wherein the alopecia areata is of the universalis type.

78. The method of claim 76, wherein the hair loss is male or female pattern alopecia.

79. The method of claim 76, wherein the hair loss is scalp or beard hair loss.

80. The method of claim 76, wherein the hydrogel comprises (i) the aldehyde-modified hyaluronic acid derivative at a weight percent (wt. %) of from 1 wt. % to 10 wt. % and (ii) the minoxidil at a wt. % of from 0.1 wt. % to 5 wt. %.

81. The method of claim 76, wherein the hydrogel comprises the polymer and the minoxidil in a 1:1 ratio.

82. The method of claim 76, wherein the sustained delivery hydrogel has chemical structure I: [chemical structure]
